(19) United States
(12) Patent Application Publication
(10) Pub. No.: US 2005/0163790 A1
(43) Pub. Date: Jul. 28, 2005

(54) Method of treatment and agents useful for same

(76) Inventors: Derek Nigel John Hart, Queensland (AU); Geoffrey Roger Hill, Queensland (AU); Kelli Patricia Anne McDonald, Queensland (AU)

Correspondence Address: Scully Scott Murphy & Presser, 400 Garden City Plaza, Garden City, NY, 11530, US

(21) Appl. No.: 10/493,451

(22) PCT Filed: Oct. 24, 2002
(86) PCT No.: PCT/AU02/01512

(30) Foreign Application Priority Data
Oct. 24, 2001 (AU) PR 8456
Oct. 16, 2002 (AU) 2002952098

(51) Int. Cl. A61K 39/00

(52) U.S. Cl. 424/185.1

(57) ABSTRACT

The present invention relates generally to a method of modulating the immunoactivity of a population of immune cells and, more particularly to a method of down-regulating the immunoactivity of an immunocompetent graft. The method of the present invention is useful, inter alia, in the treatment and/or prophylaxis of conditions characterised by aberrant, unwanted or otherwise inappropriate graft immunoactivity such as, but not limited to, the prophylaxis treatment of graft versus host disease in allogeneic stem cell graft recipients.

### FIELD OF THE INVENTION

The present invention relates generally to a method of modulating the immunoactivity of a population of immune cells and, more particularly to a method of down-regulating the immunoactivity of an immunocompetent graft. The method of the present invention is useful, inter alia, in the treatment and/or prophylaxis of conditions characterised by aberrant, unwanted or otherwise inappropriate graft immunoactivity such as, but not limited to, the prophylaxis treatment of graft versus host disease in allogeneic stem cell graft recipients.

### BACKGROUND OF THE INVENTION

Bibliographic details numerically referred to in this specification are collected at the end of the description.

The reference to any prior art in this specification is not, and should not be taken as, an acknowledgment or any form of suggestion that that prior art forms part of the common general knowledge in Australia.

Allogeneic tissue transplantation is a technique which is widely and routinely performed. In particular, allogeneic stem cell transplantation is currently indicated in the treatment of a number of malignant and non malignant diseases. However, use of the procedure is limited by its serious complications. For example, in addition to the issue of transplant rejection, patients in receipt of allogeneic tissues or cell populations which are themselves immunocompetent (e.g. bone marrow grafts, spleen transplant or stem cell grafts) run the risk of the development of graft versus host disease—a potentially fatal condition. Accordingly, there is an ongoing need to develop methods for promoting the survival of such allogeneic grafts while minimising the incidence of graft versus host disease development in the graft recipient.

In work leading up to the present invention, the inventors have determined that the pre-treatment of the graft tissue or the donor, prior to harvesting of the graft, with progenipoietin (a G-CSF and Flt-3L receptor agonist) leads to the down-regulation of graft versus host disease subsequently to allogeneic stem cell transplantation.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method of modulating the immunoactivity of an immunocompetent graft, said method comprising contacting said graft tissue with an effective amount of progenipoietin or derivative, homologue, analogue, chemical equivalent or mimetic thereof.

In another aspect there is provided a method of down-regulating the immunoactivity of an allogeneic immunocompetent graft, said method comprising contacting said graft tissue with an effective amount of progenipoietin or derivative, homologue, analogue, chemical equivalent or mimetic thereof.

In yet another aspect there is provided a method of down-regulating the immunoactivity of an allogeneic immunocompetent graft, said method comprising pre-treating said graft with an effective amount of progenipoietin or a derivative, homologue, analogue, chemical equivalent or mimetic thereof.

In yet another aspect of the present invention is directed to the generation of a population of protective immune cells, said method comprising culturing an immunocompetent population of cells with an effective amount of progenipoietin or derivative, homologue, analogue, chemical equivalent or mimetic thereof, wherein said protective immune cells down-regulate the immunoactivity of said immunocompetent cells, which immunoactivity is directed to an allogeneic target cell population.

A further aspect of the present invention relates to the use of the invention in relation to the treatment and/or prophylaxis of conditions which are characterised by the aberrant, unwanted or otherwise inappropriate immunoactivity of an allogeneic immunocompetent graft. Such immunoactivity is also referred to as graft versus host disease. The incidence of graft versus host disease can occur in any situation where an allogeneic immunocompetent graft is required to be transplanted into a recipient, such as pursuant to treatment for certain forms of cancer wherein bone marrow transplants are necessitated.

Another further aspect of the present invention contemplates a method for the prophylactic and/or therapeutic treatment of a condition characterised by the aberrant, unwanted or otherwise inappropriate immunoactivity of an immunocompetent graft, said method comprising contacting said graft tissue with an effective amount of progenipoietin or derivative, homologue, analogue, chemical equivalent or mimetic thereof, for a time and under conditions sufficient to down-regulate the immunoactivity of said graft.

In still another further aspect the present invention contemplates a method for the prophylactic and/or therapeutic treatment of a condition characterised by the aberrant, unwanted or otherwise inappropriate immunoactivity of an allogeneic immunocompetent graft, in a subject said method comprising contacting said graft tissue with an effective amount of progenipoietin or derivative, homologue, analogue, chemical equivalent or mimetic thereof, for a time and under conditions sufficient to down-regulate the immunoactivity of said graft.

In another aspect the present invention contemplates a method for the therapeutic and/or prophylactic treatment of a condition characterised by the aberrant, unwanted or otherwise inappropriate immunoactivity of an allogeneic immunocompetent graft in a subject, said method comprising administering to said mammal an effective number of protective immune cells, as hereinbefore defined, together with said graft.

Yet another aspect of the present invention relates to the protective immune cells, as defined hereinbefore, and their use in accordance with the methods previously disclosed.

### BRIEF DESCRIPTION OF THE DRAWINGS

FIG. 1 is a graphical representation of the effect of donor pretreatment on spleen phenotype. Naive B6 mice were treated with control diluent (open bars), G-CSF (10 ug/animal/day for 10 days, hatched bars), or ProGP-1 (20 ug/animal/day for 10 days, solid bars). Spleens were harvested on day 11, chopped, digested and phenotyped. DC were either CD11c^{dim}/B220^{hi} or CD11c^{hi}. (A) Proportion of lineage cells per spleen. (B) Absolute numbers of lineage cells per spleen. *P<0.05 compared to controls.

FIG. 2 is a graphical representation of the effect of cytokine pretreatment on splenic dendritic cell phenotype. Naive B6 mice were treated with control diluent, G-CSF or ProGP-1 as above. DC were enriched as described, presorted to remove autofluorescent macrophages (A), and stained with CD11c and B220 (B). The CD11c^{hi} DC (R1) from control spleen (C), G-CSF spleen (D) and ProGP-1 spleen (E) were further analysed for CD4 and CD8 expression.

FIG. 3 is a graphical representation of donor pretreatment with ProGP-1 attenuating GVHD severity. Survival curves by Kaplan-Meier analysis, pooled from two similar experiments. Donor B6 mice were treated with G-CSF (10 ug/animal/day for 10 days), ProGP-1(20 ug/animal/day for 10 days) or control diluent. Splenocytes (10⁷) from control (control allogeneic, n=15), G-CSF (G-CSF allogeneic, n=20) and ProGP-1 (ProGP-1 allogeneic, n=15) treated donors were harvested on day 11 and transplanted into lethally irradiated (1100 cGy) B6D2F1 recipient mice. Additional ProGP-1 T cells were added to a ProGP-1 (ProGP-1 allogeneic adjusted, n=13) cohort to equilibrate the T cell dose across the groups. Control treated T-cell depleted (TCD allogeneic, n=8) spleen was transplanted as non-GVHD controls. Survival: P<0.000 1 for control allogeneic versus all others, P=0.05 for G-CSF allogeneic versus ProGP-1 allogeneic.

FIG. 4 is a graphical representation of donor pretreatment with pro-GP allowing escalation of graft cell dose above that possible with donor pretreatment with G-CSF. (A and B) Survival curves by Kaplan-Meier analysis, pooled from three similar experiments. Donor B6 mice were treated as in FIG. 3. Splenocytes were harvested on day 11 and transplanted into lethally irradiated B6D2F1 recipient mice at doses of 4×10⁶/animal (for G-CSF group only, n=10), 10×10⁶/animal (n=10-20), 60×10⁶/animal (n=10-15) 100×10⁶/animal (n=10). This equates to T cell doses of 1.2×10⁶, 3.0×10⁶, 18×10⁶ and 30×10⁶ in G-CSF treated donors and 1.2×10⁶, 7.2×10⁶ and 12×10⁶ in pro-GP treated donors. P<0.03 for all G-CSF versus ProGP-1 (10×10⁶ and 60×10⁶). *P=0.26 for G-CSF (10×10⁶) versus ProGP-1(100×10⁶). (C) GVHD clinical scores as described in Methods were determined as a measure of GVHD severity in surviving animals. *P <0.05 for G-CSF (10×10⁶) vs ProGP-1 (60×10⁶) and **P<0.01 for G-CSF (10×10⁶) vs ProGP-1 (10×10⁶) at time points indicated.

FIG. 5 is a graphical representation of the effect of cytokine pretreatment on splenic T cell phenotype. Naive B6 mice were treated with control diluent, G-CSF or ProGP-1 as described in the legend to FIG. 3. Splenocytes were harvested, digested and CD3 positive T cells were examined for their expression of CD4, L-selectin, CD44 and CD25 by three colour flow cytometry.

FIG. 6 is a graphical representation of ProGP-1 expanded donor DC populations failing to confer protection from GVHD. Survival curves by Kaplan-Meier analysis, pooled from two similar experiments. Donor B6 mice were treated with ProGP-1 or control diluent. Splenocytes were harvested on day 11 and control splenocytes were transplanted into lethally irradiated B6D2F1 recipient mice at doses of 10⁷/animal (allogeneic, n=10). ProGP-1 expanded CD11c^{hi} (allogeneic+CD8 DC, n=10) or CD11c^{dim}/B220^{hi} (allogeneic+B220 DC, n=5) were and added to control splenocytes in numbers equivalent to those in unseparated ProGP-1 spleen (10⁶ CD11c^{hi} and 2.5×10⁵ CD11c^{dim}/B220^{hi}). Syngeneic spleen (syngeneic, n=3) was transplanted as non-GVHD control.

FIG. 7 is a graphical representation of ProGP-1 expanded spleen producing IL-10 and TGFβ, and inhibiting TNFα production after allogeneic SCT. Unfractionated spleen cells from control (open bars), G-CSF (shaded bars) or ProGP-1 (solid bars) treated donors were stimulated in vitro with LPS. IL-10 (A) and TGFβ (B) were determined in 48 hour culture supernatants by ELISA. Results are mean ±SD of triplicate wells and represent one of three identical experiments. (C) Animals were transplanted with whole control spleen (open bars, n=4), G-CSF spleen (shaded bars, n=4) or ProGP-1 spleen (solid bars, n=4) as in FIG. 1. Peritoneal macrophages were harvested from animals 7 days after SCT and stimulated with LPS. TNFα was determined in 5 hour culture supernatants by ELISA. Results are normalized to production per 10⁵ macrophages based on CD11b staining. P<0.05 vs control spleen. ND=not detected

FIG. 8 is a graphical representation of donor pretreatment with ProGP-1 abrogating T cell allo-reactivity in vivo. (A) Survival curves by Kaplan-Meier analysis, pooled from two similar experiments. Donor B6 mice were treated as in FIG. 3. Spenocytes were harvested on day 11 and control spleen was T-cell depleted. T cells from control (control allogeneic T, n=25), G-CSF (G-CSF allogeneic T, n=12) and ProGP-1 spleen (ProGP-1 allogeneic T, n=15) were purified and added back in equal numbers (3×10⁶) to T cell depleted control spleen (7×10⁶). T cell depleted control spleen (TCD allogeneic, n=5) was transplanted as a non-GVHD control. These grafts were transplanted into lethally irradiated B6D2F1 recipient mice. Survival: P<0.001, G-CSF B6 T versus control B6 T; P<0.0001, G-CSF allogeneic T versus ProGP-1 allogeneic T. (B) GVHD clinical scores as described in Methods were determined as a measure of GVHD severity in surviving animals. *P <0.05 between G-CSF T and ProGP-1 T curves at the time points indicated.

FIG. 9 is a graphical representation of donor pretreatment with ProGP-1 reducing GI tract injury and inflammatory cytokine generation after SCT. Recipient mice were transplanted as in FIG. 6. (A) GI tract histology was determined by semi-quantitative histology as described in Methods in recipients of control T cells (open bars, n=6), G-CSF T cells (shaded bars, n=5), ProGP-1 T cells (solid bars, n=5) or T cell depleted spleen (stippled bars, n=4). (B) 10 days after transplant, TNFα was determined in the sera of recipients of control T cells (open bars, n=7), G-CSF T cells (shaded bars, n=5), ProGP-1 T cells (solid bars, n=5) or T-cell depleted spleen (stippled bars, n=3) by ELISA as described in Methods. **P<0.0l versus control B6 T.

### DETAILED DESCRIPTION OF THE INVENTION

The present invention is predicated, in part, on the determination that progenipoietin pre-treatment of graft donors minimises the occurrence of graft versus host disease in the graft recipient subsequently to receipt of an allogeneic immunocompetent graft.

Accordingly, one aspect of the present invention is directed to a method of modulating the immunoactivity of an immunocompetent graft, said method comprising contacting said graft tissue with an effective amount of progenipoietin or derivative, homologue, analogue, chemical equivalent or mimetic thereof.

Reference to “progenipoietin” should be understood as a reference to all forms of progenipoietin and, to the extent that it is not specified, to functional derivatives, homologues, analogues, chemical equivalents or mimetics thereof. This includes, for example, all protein forms of this molecule or its functional equivalents or derivatives including, for example, any isoforms which may arise from alternative splicing of the encoding MRNA. It includes reference to functional mutants, polymorphic variants or homologues of this molecule. It also includes reference to functional analogues or equivalents of this molecule. Without limiting the present invention to any one theory or mode of action, there are six known (functional variants of progenipoietin termed progenipoietin 1-6. Accordingly, reference to “progenipoietin” should be understood to encompass reference to those 6 variants. Preferably, said progenipoietin is progenipoietin-1. Reference to “progenipoietin” should also be understood to include reference to genetic molecules encoding progenipoietin or to derivatives, homologues or analogues of said nucleic acid molecules.

Reference to an “immunocompetent graft” should be understood as a reference to a population of cells which includes immune cells. By “immune cells” is meant cells which directly or indirectly contribute to one or more aspects of an immune response such as, but not limited to, facilitating antigen presentation (e.g. dendritic cells, B cells), phagocytosis (e.g. macrophages), immune effector mechanisms (e.g. cytotoxic T cells, antibody dependent cytotoxic cells, granulocytes), antibody production (e.g. B cells), cytokine production (e.g. T helper cells, stromal cells, granulocytes). It should be understood that the subject immune cells may be at any differentiative stage. Accordingly, the cells may be immature and therefore functionally incompetent in the absence of further differentiation. In this regard, highly immature cells such as stem cells or CFU-I, which retain the capacity to differentiate into a range of immune or non-immune cell types, should nevertheless be understood to satisfy the definition of “immune cell” as utilised herein due to their capacity to differentiate into immune cells under appropriate conditions. Accordingly, a graft comprising stem cells, for example, is an immune competent graft within the scope of the present invention. It should be further understood that the immunocompetent graft of the present invention may also comprise a non-immune cell component. This would be expected, for example, where an unpurified bone marrow or spleen cell graft, for example, is the subject of transplantation, since such a graft may be expected to comprise red blood cells, fibroblasts, platelets, adipocytes and other such non-immune cells.

It should be understood that the graft which is transplanted into a recipient and which is treated in accordance with the method of the present invention may be in any suitable form. For example, the graft may comprise a population of cells existing as a single cell suspension or it may comprise a tissue sample fragment or an organ. The cells or tissues may be donated from any suitable source. For example, the cells may be isolated from an individual or from an existing cell line. The cells may be primary cells or secondary cells. A primary cell is one which has been isolated from an individual. A secondary cell is one which, following its isolation has undergone some form of in vitro manipulation such as genetic manipulation. The subject tissue graft may also be derived directly from an individual or it may be derived from an in vitro source such as a tissue sample or organ which has been generated or synthesised in vitro. The subject tissue or organ may also have been manipulated subsequently to its isolation from a donor.

The process of the invention is preferably utilised to modulate the immunoactivity of a graft which has been or is to be introduced to a recipient in an allogeneic capacity, i.e. wherein the donor is of the same species as the recipient but is MHC incompatible. The process of the present invention may also be applied in the context of a “xenogeneic” transplant meaning the donor cells were isolated from a different species to that of the recipient (for example, where pig cells are introduced into a human recipient). Preferably, the process of the present invention is applied in the context of an allogeneic transplant. In this regard, reference hereinafter to an “allogeneic” immunocompetent graft should be understood as a reference to a graft which is proposed to be utilised in the contexts of an allogeneic transplant. As detailed herein, the graft may be treated with progenipoietin subsequently to transplant to an allogeneic recipient or prior to the occurrence of this event.

More particularly, there is provided a method of down-regulating the immunoactivity of an allogeneic immunocompetent graft, said method comprising contacting said graft tissue with an effective amount of progenipoietin or derivative, homologue, analogue, chemical equivalent or mimetic thereof.

Preferably, said progenipoietin is progenipoietin-1.

Reference to the “immunoactivity” of an immunocompetent graft should be understood as a reference to the functional activity of one or more of the immune cells comprising the graft, wherein said functional activity directly or indirectly contributes to an immune response which is directed against the graft recipient. By “directed against the graft recipient” is meant that the immune response which is directly or indirectly contributed to by the immune cells of the graft is directed to rejecting one or more of the cells of the recipient, due to these cells being recognised as foreign in light of differences in MHC profiles between the donor cells of the graft and the recipient's cells.

The method of the present invention is predicated on the determination that pre-treatment of allogeneic graft tissue with progenipoietin down-regulates the anti-recipient immunoactivity which immunocompetent grafts induce subsequently to their transplantation. In this regard, it should be understood that the subject graft may be contacted with progenipoietin by any suitable means including, but not limited to:
(i) administering the progenipoietin to the graft donor, prior to removal of the graft
(ii) in vitro administration of progenipoietin to the graft tissue subsequently to its removal from a donor but prior to its transplantation. This method will be of particular importance where the immunocompetent graft is derived from stored tissues or tissues which have been generated or cultured in vitro
(iii) administration of progenipoietin to the graft recipient at or about the time of graft transplantation.

The subject pre-treatment may be achieved by any suitable means which would be well known to the person of skill in the art.

Preferably, the graft is treated with progenipoietin prior to transplantation, that is, in accordance with the method detailed in points (i) or (ii), above. In this regard, treatment of the graft with progenipoietin prior to transplantation is referred to herein as “pre-treatment”.

According to this preferred embodiment, there is provided a method of down-regulating the immunoactivity of an allogeneic immunocompetent graft, said method comprising pre-treating said graft with an effective amount of progenipoietin or a derivative, homologue, analogue, chemical equivalent or mimetic thereof.

Preferably, said progenipoietin is progenipoietin-1.

“Derivatives” include fragments, parts, portions, mutants, variants and mimetics from natural, synthetic or recombinant sources including fusion proteins. Parts or fragments include, for example, active regions of progenipoietin. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences including fusions with other peptides, polypeptides or proteins.

Chemical and functional equivalents of the progenipoietin or its encoding nucleic acid molecule should be understood as molecules exhibiting any one or more of the functional activities of these molecules and may be derived from any source such as being chemically synthesized or identified via screening processes such as natural product screening.

The derivatives of progenipoietin include fragments having particular epitopes or parts of the entire molecule fused to peptides, polypeptides or other proteinaceous or non-proteinaceous molecules.

Analogues of progenipoietin contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6tri-itrobenzene sulphonic acid (TNBS); acylation of amino groups with succinic anhydride and tetrahydrophthalic anhydride; and pyridoxylation of lysine with pyridoxal-5-phosphate followed by reduction with NaBH4.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpencillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homobifunctional crosslinkers such as the bifunctional imido esters having (CH₂).ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Reference to “down-regulating” the immunoactivity of the subject immunocompetent graft should be understood as a reference to at least partially down-regulating said activity. It should be understood however, that the overall down-regulation of this activity may be mechanistically achieved by up-regulating the activity of certain protective immune cells.

An “effective amount” or an “effective number” means an amount or number necessary to at least partly obtain the desired response, or to delay the onset or inhibit progression of halt altogether, the onset or progression of a particular condition being treated. The amount varies depending upon the health and physical condition of the individual to be treated, the taxonomic group of the individual to be treated, the degree of protection desired, the formulation of the composition, the assessment of the medical situation and other relevant factors. It is expected that the amount will fall in a relatively broad range which can be determined through routine trials.

In this regard, without limiting the present invention to any one theory or mode of action, the inventors have determined that progenipoietin pre-treatment of grafts up-regulates the proliferation and differentiation of protective immune cells including, but not limited to, CD4⁺ T cells which are protective against graft versus host disease. Accordingly, the present invention should be understood to extend to the generation of a protective donor immune cell population and to the administration of these immune cells, either prior to, subsequently to or concomitantly together with a donor derived immunocompetent graft, to a recipient.

Accordingly, another aspect of the present invention is directed to the generation of a population of protective immune cells, said method comprising culturing an immunocompetent population of cells with an effective amount of progenipoietin or derivative, homologue, analogue, chemical equivalent or mimetic thereof, wherein said protective immune cells down-regulate the immunoactivity of said immunocompetent cells, which immunoactivity is directed to an allogeneic target cell population.

Preferably, said progenipoietin is progenipoietin-1 and said immuncompetent population of cells is a stem cell population, bone marrow population or spleen cell population.

Preferably, said population of protective immune cells is a population of protective CD4⁺ T cells. Said protective immune cells may be optionally purified from the subject culture.

Reference to “protective immune cells” herein should be understood as a reference to cells which have been generated pursuant to progenipoietin treatment and which function to down-regulate the immunoactivity of immunocompetent cells which are syngeneic relative to the protective cells but allogeneic relative to the target cell population which is the subject of protection.

Reference herein to “dendritic cells” should be read as including reference to cells exhibiting dendritic cell morphology, phenotype or functional activity and to mutants or variants thereof. The morphological features of dendritic cells may include, but are not limited to, long cytoplasmic processes or large cells with multiple fine dendrites. Phenotypic characteristics may include, but are not limited to, expression of one or more of MHC class I, MHC class II, CD1 or CD8. Functional activity includes but is not limited to, a stimulatory capacity for naive allogeneic T cells. “Variants” include, but are not limited to, cells exhibiting some but not all of the morphological or phenotypic features or functional activities of dendritic cells. “Mutants” include, but are not limited to, dendritic cells which are transgenic wherein said transgenic cells are engineered to express one or more genes such as genes encoding antigens, immune modulating agents or cytokines or receptors. Preferably, said dendritic cell is a lymphoid dendritic cell and, even more particularly, a CD8^{HI/DIM} dendritic cell.

Without limiting the present invention to anyone theory or mode of action, the protective CD4⁺ T cell population is thought to be a population of Th3 type cells.

The donor and recipient of the subject invention are mammals and include, humans, primates, livestock animals (e.g. sheep, pigs, cattle, horses, donkeys), laboratory test animals (e.g. mice, rabbits, rats, guinea pigs), companion animals (e.g. dogs, cats) and captive wild animals (e.g. foxes, kangaroos, deer). Preferably, the mammal is a human. Although the present invention is exemplified herein with respect to laboratory test animals, this should not be understood in any way as limiting the application of the present invention to humans.

A further aspect of the present invention relates to the use of the invention in relation to the treatment and/or prophylaxis of conditions which are characterised by the aberrant, unwanted or otherwise inappropriate immunoactivity of an allogeneic immunocompetent graft. Such immunoactivity is also referred to as graft versus host disease. The incidence of graft versus host disease can occur in any situation where an allogeneic immunocompetent graft is required to be transplanted into a recipient, such as pursuant to treatment for certain forms of cancer wherein bone marrow transplants are necessitated.

Accordingly, another aspect of the present invention contemplates a method for the prophylactic and/or therapeutic treatment of a condition characterised by the aberrant, unwanted or otherwise inappropriate immunoactivity of an immunocompetent graft, said method comprising contacting said graft tissue with an effective amount of progenipoietin or derivative, homologue, analogue, chemical equivalent or mimetic thereof, for a time and under conditions sufficient to down-regulate the immunoactivity of said graft.

More particularly, the present invention contemplates a method for the prophylactic and/or therapeutic treatment of a condition characterised by the aberrant, unwanted or otherwise inappropriate immunoactivity of an allogeneic immunocompetent graft, in a subject said method comprising contacting said graft tissue with an effective amount of progenipoietin or derivative, homologue, analogue, chemical equivalent or mimetic thereof, for a time and under conditions sufficient to down-regulate the immunoactivity of said graft.

Preferably, said progenipoietin is progenipoietin-1.

More preferably, said condition is graft versus host disease.

Still more preferably said graft is a bone marrow graft, spleen cell graft or a stem cell graft.

Even more preferably, said graft is pre-treated with said progenipoietin. In another aspect the present invention contemplates a method for the therapeutic and/or prophylactic treatment of a condition characterised by the aberrant, unwanted or otherwise inappropriate immunoactivity of an allogeneic immunocompetent graft in a subject, said method comprising administering to said mammal an effective number of protective immune cells, as hereinbefore defined, together with said graft.

Preferably, said condition is graft versus host disease.

Still more preferably, said protective immune cells are derived from a progenipoietin-1 treated bone marrow population, spleen cell population or a stem cell population and said protective immune cells are CD4⁺ T cells. Even more preferably, said graft is a bone marrow graft, spleen cell graft or stem cell graft.

The subject protective immune cells and graft are preferably co-administered. By “co-administered” is meant simultaneous administration in the same formulation or in different formulations via the same or different routes or sequential administration via the same or different routes. By “sequential” administration is meant a time difference of from seconds, minutes, hours or days between the transplantation of the graft and the administration of the protective immune cells.

Preferably the graft and the protective immune cells are co-administered.

Without limiting the present invention in any way, the down-regulation of allogeneic immunocompetent graft immunoactivity now facilitates the administration of higher concentrations of graft cells to a recipient.

Reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition. Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

The present invention further contemplates a combination of therapies, such as the administration of the subject pre-treated graft together with a low dose of immunosuppressive drugs.

Yet another aspect of the present invention relates to the protective immune cells, as defined hereinbefore, and their use in accordance with the methods previously disclosed.

The present invention is further defined by the following non-limiting examples:

### EXAMPLE 1

### Donor Pretreatment With Progenipoitin-1 is Superior to G-CSF in Preventing Graft-vs-Host Disease After Allogenic Tem Cell Transplantation

### Materials & Methods

Mice

Female C57BL/6 (B6, H-2^{b}, Ly-5.2⁺), B6 PTRCA Ly-5^{a} (H-2^{b}, Ly-5. 1−) and B6D2F1 (H-2^{b/d}, Ly-5.2⁺) (Morse, H. C., Shen, F. W., Hamerling, U., Immunogenetics 25, 71, 1987) mice were purchased from the Australian Research Centre (WA, Australia). The age of mice used as BMT recipients ranged between 8 and 14 weeks. Mice were housed in sterilized microisolator cages and received filtered water and normal chow, or autoclaved drinking water for the first two weeks post BMT.

Cytokine Treatment

Recombinant human G-CSF (Amgen, Thousand Oaks, Calif.), Progenipoietin (Pharmacia, St Louis, Mo.) or control diluent was diluted in 1 μg/ml or murine serum albumin in PBS before injection. Mice were injected subcutaneously with G-CSF (10 μg/animal/day), ProGP-1 (20 μg/animal/day) or diluent from day −10 to day −1.

Bone Marrow Transplantation

Mice were transplanted according to a standard protocol as has been described previously (Pan L., Delmonte J., Jalonen C. K., Ferrara J. L. M., Blood. 86, 44224429, 1995; Pan L., Teshima T., Hill G. R., Bungard D., Brinson Y. S., Reddy V. S., Cooke K. R., Ferrara J. L. M., Blood 93, 40714078, 1999). Briefly, on day −1, B6D2F1 mice received 1100 total body irradiation (¹³⁷Cs source at 108 cGy/min), split into two doses separated by 3 hours to minimize gastrointestinal toxicity. Donor spleens were chopped, digested in collagenase and DNAse, then whole unseparated spleen cells were resuspended in 0.25 ml of Leibovitz's L-15 media (Gibco BRL, Gaithersburg Md.)) and injected intravenously into recipients. In most experiments, PTRCA Ly-5^{a} (H-2^{b}, Ly-5.1⁺) animals were used as donors (see below). Survival was monitored daily, recipient's body weights and GVHD clinical score were measured weekly. Donor cell engraftment was determined by examining the proportion of Ly-5.1⁺/Ly-5.2⁺+Ly-5.1⁺ cells in peripheral blood or spleen after transplantation.

Assessment of GVHD

The degree of systemic GVHD was assessed by a scoring system which sums changes in five clinical parameters: weight loss, posture (hunching), activity, fur texture and skin integrity (maximum index=10) (Hill G.e, Cooke K. R., Teshima T., Crawford J. M., Keith J. C. J., Brinson Y. S., Bungard D., Ferrara J. L. M. J. Clin. Invest. 102, 115-123, 1998; Cooke K. R-, Kobzik L., Martin T. R., Brewer J., Delmonte J., Crawford J. M., Ferrara J. L. M., Blood. 88, 3230-3239, 1996; Hill G. R., Crawford J. M., Cooke K. R, Brinson Y. S., Pan L., Ferrara J. L. M. (1997) Blood 90, 3204-3213; Hill R. G. Teshima T., Gerbita A., Pan L., Cooke K. R., Brinson Y. S., Crawford J. M., Ferrara J. L. M., J. Clin. Invest. 104, 459-467, 1999). Individual mice were ear-tagged and graded weekly from 0 to 2 for each criterion without knowledge of treatment group. Animals with severe clinical GVHD (scores >6 were sacrificed according to ethical guidelines and the day of death deemed to be the following day).

Splenocyte and Dendritic Cell Preparation

Dendritic cell purification was undertaken as previously described (Vremec D., Pooley J. Hochrein H., Wu L., Shortman K., J. Immunol. 164, 2978, 2000). Briefly, spleens were chopped and digested in collagenase and DNAse. Light-density cells were selected by nycodenz density (1.077 g/l) centrifugation. Non DC-lineage cells were depleted by coating with rat IgG antibodies to B cells (CD19), T cells (CD3, Thy1), granulocytes (Gr-1) and erythroid cells (Ter-119). The coated cells were then removed by magnetic beads coupled to anti-rat IgG (Dynal ASA, Oslo, Norway). In some experiments, myeloid (CD4⁺) DC were also removed by the addition of anti-CD4 (GK1.5). At the end of this procedure, 65-85% of these cell populations were DC (class II.DC11c^{hi}). DC were presorted to remove autofluorescent macrophages (prior to phenotypic analysis) and then FACS sorted (FACSvantage, BD) to >98% purity using phycoerythrin (PE) CD11c and PE-Cy5 B220 staining.

T Cell Depletion

Splenocytes were depleted of T cells by incubation for 40 minutes (4 degrees) with hybridoma supernatants containing CD4 (2.43), CD8 (3.155) and Thy1.2 (HO-13-4). Cell suspensions were then incubated with rabbit complement (Cederlane Laboratories, Ontario, Canada) for 30 minutes at 37 degrees and the process repeated. Resulting cell suspensions had <1% contaminating viable CD3 T cells.

FACS Analysis

Fluorescein isothiocyanate (FIFC) conjugated monoclonal antibodies (mAb) to mouse Ly 5.1 and Ly 5.2 antigens, FITC conjugated CD4, CD8, 11c, class II, CD3, GF-1, 11b, B220 and identical PE conjugated antibodies were purchased from PharMingen (San Diego, Calif.). In DC analysis, CyChrome CD4 and CD8 antibodies were also used from Pharmingen (San Diego, Calif.). Cells were first blocked with mAb 2.4G2 for 15 minutes at 4° C., then with the relevant conjugated mAb for 30 minutes at 4° C. Finally, cells were washed twice with PBS/0.2% BSA, fixed with PBS/1% paraformaldehyde and analyzed by FACScalibur (Becton Dickinson, San Jose, Calif.). Propodium iodide was added in the final wash to label dead cells. Dendritic cell staining was undertaken on presorted cell populations in which autofluorescent cells were removed by high speed pre-sorting (FACSvantage) and subsequent analysis was performed the same day on unfixed cells.

Cell Cultures

Culture media additives were purchased from Gibco BRL (Gaithersburg, Md.) and media was purchased from Sigma (St Louis, Mo.). Peritoneal macrophages were lavaged and pooled from individual animals within a treatment group before culture at 1×10⁵ cells per well in flat bottomed 96 well Falcon plates (Lincoln Park, N.J.) with or without LPS. cell culture was performed in 2% FCS/DMEM (day 7 cultures) supplemented with 50 units/ml penicillin, 50 μg/ml streptomycin, 2 mM L-glutamine, I mM sodium pyruvate, 0.1 mM non-essential amino acid, 0.02 mM β-mercaptoethanol, and 10 mM HEPES, pH 7.75 at 37° C. in a humidified incubator supplemented with 5% CO₂. Supernatants were collected at 5 hours for TNF α analysis by ELISA. Peritoneal macrophages lavaged from animals 7 days after transplant were >95% donor as determined by 5.1 staining Remaining cell culture was performed in 10% FCS/DMEM. In in vitro experiments, purified B6 T cells were cultured in round bottom 96 well plates (Falcon, Lincoln Park, N.J.) with 10⁵ irradiated (2000Rad) F1 peritoneal macrophages (primary MLC) and supernatants harvested at 72 hours. Cultures were then pulsed with ³H-thymidine (1 μCi per well) and proliferation was determined 16 hrs later on a 1205 Betaplate reader (Wallac, Turku, Finland). In secondary MLC, purified T cells were cultured in flat bottom 24 well plates (Falcon, Lincoln Park, N.J.) with irradiated (2000Rad) splenocytes. Six days later, cells were removed and restimulated with F1 macrophages. Supernatants were removed 24 hrs later and ³H-thymidine added as above. In experiments of T cell function ex vivo, splenocytes were removed from animals 7-10 days after transplant and 3-6 spleens combined from each group. These cells were plated in 96 well flat bottomed plates with platebound CD3 and CD28 (both 10 μg/ml) or 10⁵ irradiated (2000 Rad) peritoneal macrophages lavaged from naive F1 (allogeneic) animals. At 40 hours, cultures were pulsed with ³H-thymidine (1 μCi per well) and proliferation was determined 16 hrs later. In separation experiments, CD4+cells were positively selected from splenocyte populations using the mini-MACS system (Miltenyi Biotech, Bergisch Gladbach, Germany) or Fluorescent Activated Cell Sorting (FACSvantage, BD). Following selection, positive and negative fractions were FACS stained and each fraction has <1% contamination of opposing CD4+or CD8+cells. Purified CD4+or CD8+populations were then plated and analyzed as above.

⁵¹Cr Release Assays

2×10⁶ P815 (H-2^{d}) or EL4 (H-2^{b}) tumor targets were labelled with 100 μCu of ⁵¹Cr for 2 hours. After washing three times, labelled targets were plated at 10⁴ cells per well in U bottom plates (Costar, Cambridge, Mass.). CD8+splenocytes from allogeneic BMT recipients (prepared by magnetic selection as described above) were added to triplicate wells at varying effector to target ratios and incubated for 5 hours in the presence of IL-2 (10 U/ml). Maximal and background release was determined by the addition of Triton-X (Sigrna, St Louis, Mo.) or media alone to targets respectively. ⁵¹Cr activity in supernatants taken 5 hrs later were determined in a scintillation counter and lysis was expressed as a percentage maximum. Lysis was expressed in lytic units (1000/effector: target ratio that induced 10 and 20% lysis).

Cytokine ELISAS

The antibodies used in the TNF α, IFN}, IL-10, TGFβand IL4 assays were purchased from PharMingen (San Diego, Calif.). All assays were performed according to the manufacturer's protocol. briefly, samples were diluted 1:3 to 1:24 and TNF α, IFNγ, IL-10, TGFβ and IL-4 proteins were captured by the specific primary monoclonal antibody (mAb), and detected by biotin-labelled secondary mAb followed by HRP-conjugated streptavidin. The biotin-labelled assays were developed with TMB substrate (Kirkegaard and Perry laboratories, Gaithersburg, Md.). Plates were read at 450 nm using a microplate reader (Bio-Rads Labs, Hercules, Calif.). Recombinant cytokines (PharMingen) were used as standards for ELISA assays. Samples were run in duplicate and the sensitivity of the assays was 16 to 20 pg/ml for TNF α, 0.063 U/ml for IFNγ, and 15 pg/ml for IL-10 and IL-4. Supernatants were collected after 4-5 hours of culture for TNF α, 40 hours for IL-4, IL-10 and IFNα analysis. Serum was stored at −70° C. until analysis. TNF a from in peritoneal cells is expressed as pg per 10⁵ macrophages, as previously described (Hill et al., 1997, supra).

Histology

Formalin-preserved distal small bowel was embedded in paraffin, and 5 μm thick sections were stained with haematoxylin and eosin for histologic examination. Slides were coded and examined in a blinded fashion by one individual (ADC), using a semi-quantitative scoring system for abnormalities known to be associated with GVHD (Hill et al., 1998, supra; Hill et al., 1997, supra; Krijanovsky, O. I., Hill, G. R., Cooke, K. R., Teshima, T., Brinson, Y. S., Ferrara, J. L. M. Blood (1999); 94:825-31). Specifically, seven parameters each were scored for small bowel (villous blunting, crypt regeneration, crypt epithelial cell apoptosis, crypt loss, luminal sloughing of cellular debris, lamina propria inflammatory cell infiltrate, and mucosal ulceration). The scoring system for each parameter denoted 0 as normal; 0.5 as focal and rare; 1 as focal and mild; 2 as diffuse and mild; 3 as diffuse and moderate; and 4 as diffuse and severs, as previously published in human (Snover, D. C., Weisdorf, S. A., Ramsay, N. K., McGlave, P., Kersey, J. H. Hepatology (1984); 4:123-130; Snover, D. C., Weisdorf, S. A., Vercellotti, G. M., Rank, B., Hutton, S., McGlave, P. Human Pathol. (1985); 16:387-392) and experimental (Hill et al., 1998, supra; Hill et al., 1997, supra; Krijanovski et al., 1999, supra) GVHD histology. Scores were added to provide a total score of 28.

Statistical Analysis

Survival curves were plotted using Kaplan-Meier estimates and compared by log-rank analysis. The Mann Whitney-U test was used for the statistical analysis of cytokine data and clinical scores. P<0.05 was considered statistically significant.

### EXAMPLE 2

ProGP-1 Pretreatment Results in a Marked Expansion of CD8^{hi} DC

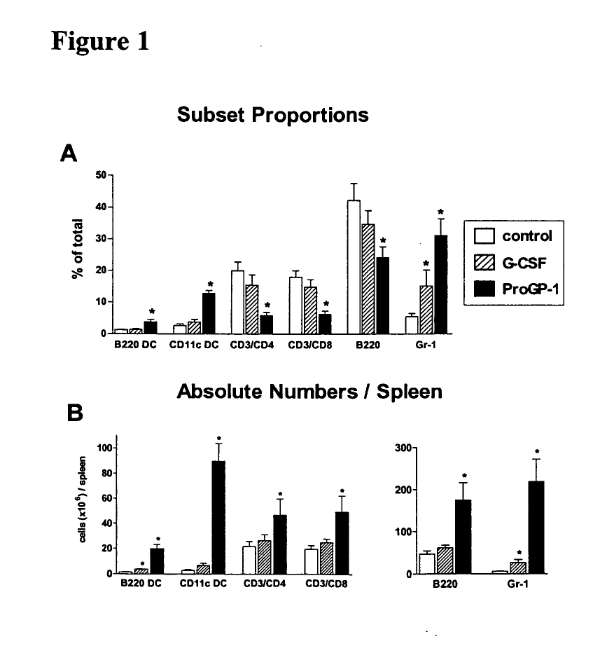

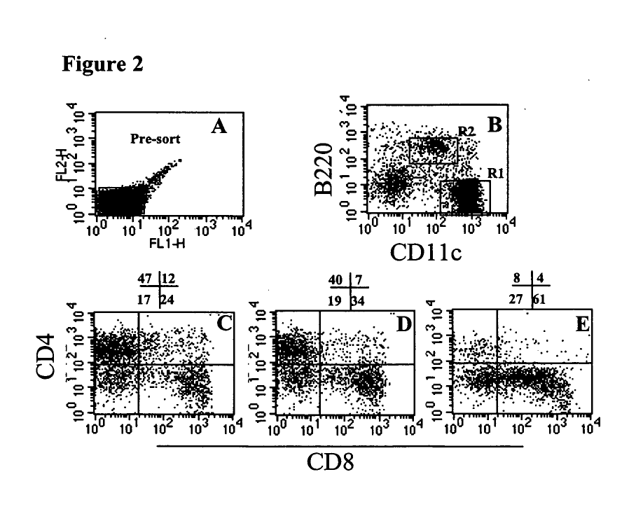

In these studies the effect on GVHD of donor pretreatment with ProGP-1 and G-CSF was compared, the latter being the current cytokine used for the mobilization of allogeneic stem cells in clinical practice. Injections of control diluent, ProGP-1 (20 ug/animal) or G-CSF (10 ug/animal) were administered daily for 10 days. This administration regimen for ProGP-1 was shown in preliminary studies to result in the greatest expansion of stem cells and DC, consistent with recent reports. The G-CSF dose was half that of the chimeric G-CSF/FLT-3L molecule ProGP-1. At the end of this treatment period, absolute numbers of splenocytes increased by 65% in the G-CSF group and 700% in the ProGP-1 group. As shown in FIG. 1, the percentages of DC, CD4 and CD8 T cells and B cells were similar in control and G-CSF treated animals, consistent with our previous findings.²⁰ In contrast, ProGP-1 resulted in a 10-fold increase in the percentage of CD11c^{hi} and CD11c^{dim}/B220^{hi} DC (FIGS. 1 and 2B) and a 65% reduction in the percentage of T cells. Significant (30%) reductions in the overall proportion of B220⁺/CD19⁺ cells were also noted following ProGP-1 administration. As expected, the proportion of granulocytes was significantly increased in both G-CSF and ProGP-1 treated animals. To examine the profound effect of ProGP-1 on DC expansion more closely, DC (CD11c^{hi}) were purified and phenotyped according to the expression of CD8 and CD4, as previously published.²⁴ The percentage of CD11c^{hi}/CD8^{hi} DC increased in animals treated with either ProGP-1 or G-CSF compared to those treated with control diluent (FIG. 2). This was most dramatic following ProGP-1 administration, which resulted in a 14-fold increase in the proportion of splenic CD8 DC and a 100-fold increase in the absolute number of these cells. The DC in the ProGP-1 treated donors included a CD8^{dim} subset which were all CD11b^{lo} (relative to CD4 DC CD11b expression) and a larger CD8^{hi} subset, the majority of which were also CD11b^{lo} (75%). The remaining 25% were CD11b^{neg}. Identical cellular proportions and expansion was seen in the peripheral blood of ProGP-1 treated animals, confirming that the spleen phenotype was representative of that in the blood.

### EXAMPLE 3

### Donor Pretreatment with ProGP-1 is Superior to G-CSF in Reducing the Severity of GVHD

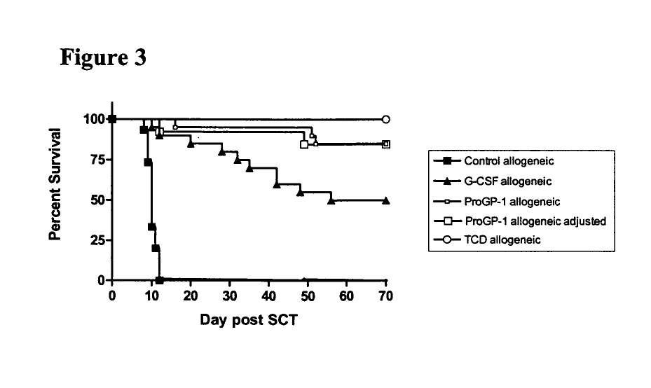

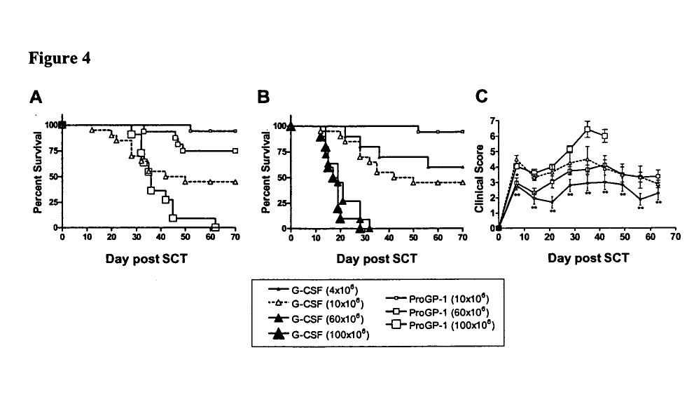

The effects of ProGP-1 mobilization were examined in a well-established murine SCT model (B6 Ly5^{a}→B6D2F1) that induces GVHD to major and minor histocompatibility antigens. Although this model utilizes spleen as a stem cell source rather than peripheral blood, its' validity has been proven by the informative data indicating beneficial effects of G-CSF on both GVHD and GVL that has since been confirmed clinically. In preliminary experiments, it was confirmed that the prolonged 10 day course of G-CSF was at least equivalent in preventing GVHD as the standard 6 day course used in clinical practice. Survival at day 70 was 50% versus 30% in recipients of splenocytes from donors treated with 10 days (n=10) versus 6 days (n=10) of G-CSF (P=0.49). In addition, clinical scores were not statistically different in the first 50 days after transplant (P>0. 12) although there was a trend to less GVHD in recipients of the 10 day course of G-CSF at later time points. Pretreatment of donors with 10 days of G-CSF was therefore used as the relevant control to ProGP-1 in subsequent experiments. In these experiments, allogeneic donor B6 animals received daily injections of either control diluent, G-CSF or ProGP-1 and splenocytes were harvested on day 11. B6D2F1 recipient mice were irradiated with 1100 cGy of TBI and transplanted with 10⁷ splenocytes from respective donors. To compensate for the reduced T cell dose in the ProGP-1 recipients, a further cohort of recipients was transplanted with ProGP-1 splenocytes in which additional purified ProGP-1 T cells were added, so as to equilibrate T cell dose (3×10⁶ T) across groups. As shown in FIG. 3 and as previously described, GVHD induced in this model is severe with all recipients of control splenocytes dying in two weeks with characteristic features of GVHD (weight loss, hunching, fur ruffling, etc). In contrast, 100% of non-GVHD controls transplanted with T cell depleted allogeneic splenocytes survived, confirming that this splenocyte dose contained sufficient stem cells to rescue lethally irradiated recipients and that GVHD is mediated by donor T cells. Allogeneic SCT recipients of G-CSF splenocytes had a significantly improved survival at day 70 compared to recipients of allogeneic control splenocytes (50% v 0%, P<0.001). Recipients of ProGP-1 splenocytes had a survival at day 70 in excess of 90% which was significantly better (P<0.05) than recipients of G-CSF splenocytes. The addition of further T cells to ProGP-1 splenocytes did not increase GVHD mortality (FIG. 3), although clinical GVHD scores were significantly increased (data not shown). In order to further establish the magnitude of protection afforded by ProGP-1 mobilization over that seen with G-CSF, cohorts of animals were transplanted with escalating doses of splenocytes from either ProGP-1 or G-CSF treated donors. As demonstrated in FIG. 4A, survival in recipients of 10⁶ splenocytes (1.2×10⁶T cells) from ProGP-1 treated donors was superior to that in recipients of splenocytes doses from G-CSF donors that ranged from 4×10⁶ to 100×10⁶(1.2×10⁶ to 30×10⁶T cells). As expected, GVHD mortality was dependent on splenocyte dose in both groups. As shown in FIG. 4B, survival in recipients of 60×10⁶ ProGP-1 treated splenocytes (7.2×10⁶T cells) was superior to that seen in recipients of 10×10⁶ G-CSF splenocytes (3×10⁶T cells; 75% v 40%, P<0.03). Survival was similar, however, when a dose of 100×10⁶ ProGP-1 splenocytes (12×10⁶T cells) was compared to 10×10⁶ G-CSF splenocytes (3×10⁶ T cells; P=0.26). In addition GVHD clinical scores (FIG. 4C) were similar in surviving recipients of 10×10⁶ G-CSF splenocytes (3×10⁶ T cells) and 60×10⁶ ProGP-1 treated splenocytes (7.2×10⁶T cells). Given the differences in T cell doses that this represents, these data suggest that donor pretreatment with ProGP-1 allows a two to four-fold escalation in T cell dose over that possible with G-CSF.

Donor T cell engraftment in the spleen 7 days after SCT was 94.7% ±1.4% in recipients of control splenocytes, 95.4% ±0.7% in recipients of G-CSF splenocytes and 96.5% ±0.1% in recipients of ProGP-1 splenocytes. The proportion of donor cells in the peripheral blood of recipients of G-CSF and ProGP-1 splenocytes at day 75 after SCT was 99.4% ±0.6 and 99.2% ±0.4% respectively. In recipients of T cell depleted splenocytes, 81% ±3.2% of peripheral blood cells were donor (P<0.05 vs G-CSF and ProGP-1), confirming that the prolonged survival following allogeneic G-CSF and ProGP-1 treated splenocytes was not due to the presence of stable mixed donor-host chimerism.

### EXAMPLE 4

### Donor Pretreatment with ProGP-1 Results in a T Cell Phenotype with Reduced Capacity to Induce GVHD

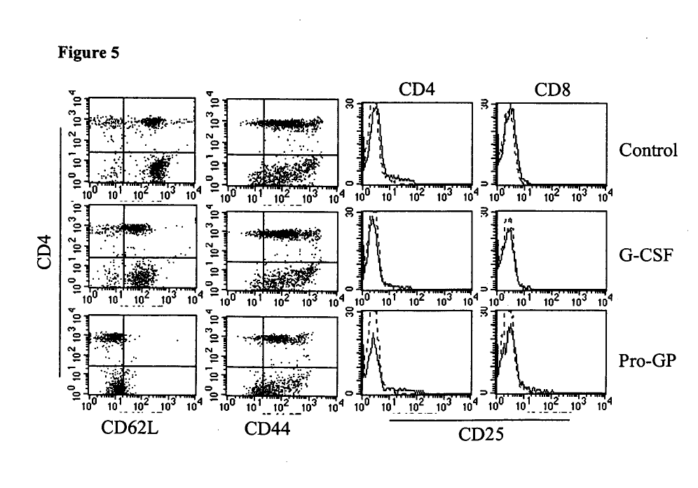

The GVHD induced in these models is dependent on T cell function and therefore the effect of G-CSF and ProGP-1 administration on T cell phenotype and function was examined. CD3⁺CD4⁺ and CD3⁺CD8⁺ T cells from ProGP-1 treated donors demonstrated an almost complete loss of L-selectin expression while T cells from G-CSF treated animals demonstrated an intermediate pattern of L-selectin loss (FIG. 5). A similar pattern of expression was demonstrated in T cells from the peripheral blood of G-CSF and ProGP-1 treated donors (data not shown). The reduction in L-selectin expression did not coincide with an increase in the proportion of CD44^{hi} T cells (FIG. 5), suggesting that the loss of L-selectin was not due to an expansion of memory T cells. In this regard, ProGP-1 and G-CSF did not induce T cell activation as assessed by CD25 (FIG. 5) and CD69 expression (data not shown). L-selectin and CD44 expression on splenic T cells from recipients of control and ProGP-1 splenocytes four days after transplant was equivalent (40% and 90% respectively), indicating that the loss of expression of these molecules prior to transplant was transient. In studies of T cell function, CD3⁺CD4⁺T cells were purified as described and stimulated in vitro with mitogen. As shown in Table 1, cytokine treatment did not alter proliferative responses although both ProGP-1 and G-CSF significantly increased the production of the type 2 cytokines IL-4 and IL-10 while IFNγ production was unchanged. To study T cell responses to alloantigen in vivo after SCT, animals were transplanted with splenocytes from control, G-CSF or ProGP-1 treated donors as in FIG. 1. Donor CD4 and CD8 T cells were purified from the spleen of animals seven days later. As shown in Table 2, CD4 T cells isolated from allogeneic SCT recipients of ProGP-1 (and to a lesser extent G-CSF) treated splenocytes failed to proliferate to host antigen. Cytokine generation (IFNγ, IL4 and IL-10) was also impaired. This impairment in proliferation was not corrected by the addition of exogenous IL-2 (50 U/ml) to MLC (control, 123,963 cpm ±11,289 cpm; G-CSF, 31,382 cpm ±1991 cpm; ProGP-1, 28,832 cpm ±2368 cpm). However, cytotoxicity to host antigens in the donor CD8 population was not significantly altered (Table 2). The difference in T cell responses before and after SCT suggests that ProGP-1 and G-CSF modulate T cell function predominantly in vivo, perhaps due to impaired T cell homing and/or the effects of additionally expanded donor cellular fractions or the products thereof.

### EXAMPLE 5

### Neither CD11c^{hi} Nor CD11c^{dim}/B220^{hi} Donor DC From ProGP-1 Treated Animals Provide Protection From GVHD

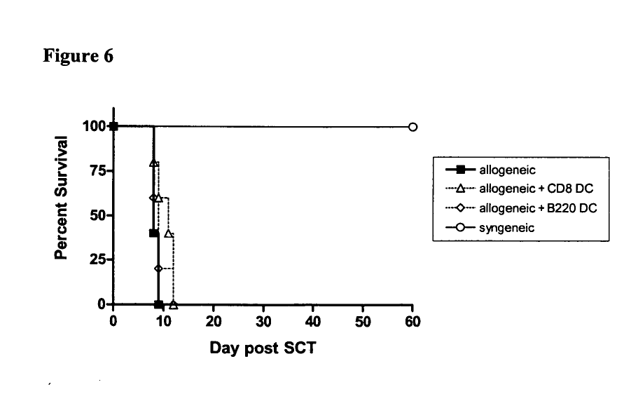

The ability of highly purified donor DC to provide protection from GVHD was examined. Aniimals were transplanted with control treated B6 spleen supplemented with FACS sorted (>98% pure) CD11c^{hi} splenic DC (predominantly CD8 positive) or CD11 c^{dim}/B220^{hi} DC from ProGP-1 treated donors in numbers that reflected the proportion present in whole ProGP-1 treated spleen (see FIG. 1). As shown in FIG. 6, all animals that received these cell populations died at a similar rate to control animals, suggesting that neither population provided protection from GVHD in isolation.

### EXAMPLE 6

### PROGP-1 Augments IL-10 and TGFB Production and Reduces TNFα Generation in Vitro

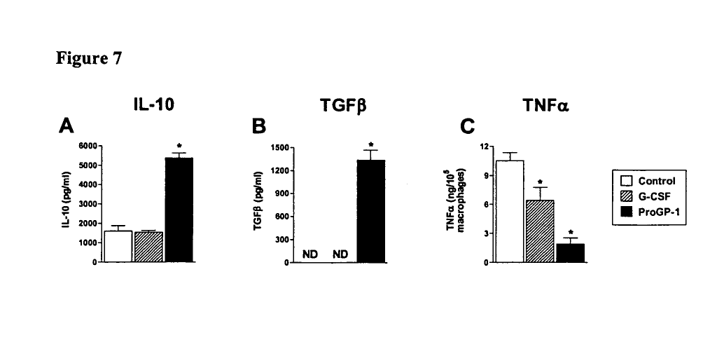

T cell function may be altered in vivo by both pro-inflammatory and anti-inflammatory cytokines, which are known to play critical roles in GVHD.^{22,32} To examine cytokine production from control, G-CSF or ProGP-1 treated donors, unseparated spleen cells were stimulated in vitro with LPS and IL-10 and TGFβ determined in culture supernatants. As shown in FIG. 7, ProGP-1 spleen produced high amounts of IL-10 and TGFβ relative to control and G-CSF spleen. After transplant with these cell populations, a major reduction in TNFα was demonstrated in cultures of macrophages from recipients of ProGP-1 treated splenocytes (FIG. 7C). Macrophages from recipients of G-CSF treated splenocytes produced intermediate quantities of TNFα. These data confirm that donor pretreatment with ProGP-1 results in a graft composition favouring anti-inflammatory cytokine production.

### EXAMPLE 7

### The Inhibition of GVHD Following Donor Pretreatment With ProGP-1 is Mediated Through Effects on The T Cell

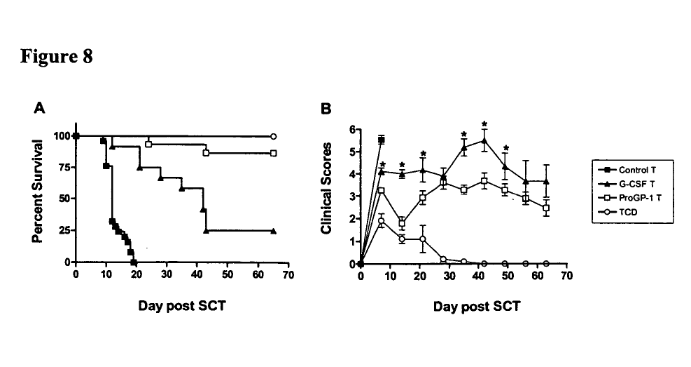

Since GVHD is a T cell dependent process, it was determined whether the ability of ProGP-1 to reduce GVHD was mediated through effects on the donor T cell. To compare the capacity of the T cells from treated animals to induce GVHD in isolation, all transplant recipients received T cell-depleted control splenocytes together with equivalent numbers of purified splenic T cells from either control, G-CSF or ProGP-1 treated donors. As shown in FIG. 8A, 100% of recipients of control T cell-depleted splenocytes survived whilst 100% of recipients of control T cell-depleted splenocytes supplemented with purified control T cells died of GVHD by day 20. In contrast, the median survival was increased in recipients of control T cell-depleted splenocytes supplemented with purified G-CSF treated T cells to 40 days and survival at day 70 increased to 25% (P<0.001 versus recipients of control T cells). Recipients of control T cell-depleted splenocytes supplemented with purified ProGP-1 treated T cells had significantly less GVHD than either group with 90% survival at day 70 (P<0.0001 versus recipients of G-CSF and control T cells). GVHD severity in surviving animals, as determined by clinical score was also significantly reduced in recipients of purified ProGP-1 treated T cells relative to purified G-CSF treated T cells (FIG. 8B). These data indicate that both G-CSF and ProGP-1 alter the capacity of donor T cells to induce GVHD and that the enhanced survival of recipients of proGP-1 treated allografts relates to T cell effects.

### EXAMPLE 8

### T Cells From ProGP-1 Treated Donors Fail to Induce GI Tract Injury and Systematic TNF α Production After Allogenetic Sct

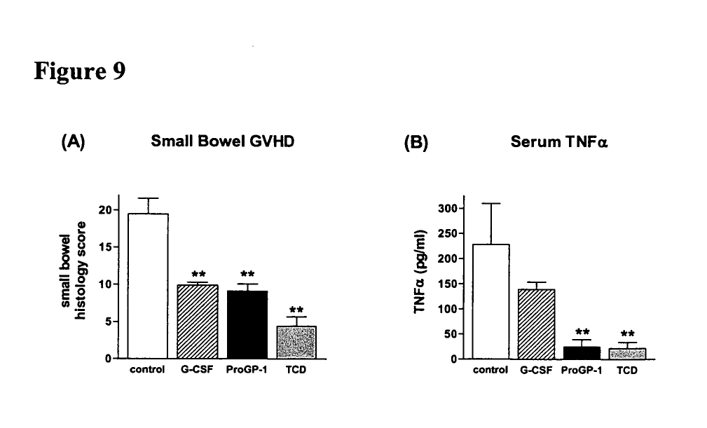

In order to confirm that the ex vivo data in Table II were representative of T cell function in vivo, IFNγ levels were determined in the sera of animals 5 days after transplant. IFNγ levels were significantly reduced in recipients of both G-CSF and ProGP- 1 treated T cells (63±6.4 U/ml vs 46.4±8.0 U/ml and 44.4±5.3 U/ml), consistent with the ex vivo data. GVHD mortality in this transplant model is TNFα dependent²² and IFNγ primes mononuclear cells to produce high TNFα levels following stimulation with bacterial derived antigens that are primarily derived from the GI tract. As shown in FIG. 9A, T cells from ProGP-1 and G-CSF treated donors failed to induce severe GVHD of the GI tract relative to recipients of control treated T cells. In vivo, TNFα levels in the sera of recipients of ProGP-1 T cells were 10-fold lower than those in recipients of control T cells and were indistinguishable from non-GVHD controls (FIG. 9B). Recipients of G-CSF treated T cells had TNFα levels intermediate between recipients of control and ProGP-1 T cells, consistent with the mortality seen in this group.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Arpinati, M., Green C. L., Heimfeld, S., Heuser, J. E., Anessetti, C. (2000) Blood. 95:2484-2490
Cooke K. R., Kobzik L., Martin T. R., Brewer J., Delmonte J., Crawford J. M., Ferrara J. L. M. (1996) Blood. 88, 3230-3239
Hill G. R, Crawford J. M., Cooke K. R., Brinson Y. S., Pan L., Ferrara J. L. M. (1997) Blood. 90, 3204-3213
Hill G. R, Cooke K. R, Teshirna T., Crawford J. M., Keith J. C. J., Brinson Y. S., Bungard D., Ferrara J. L. M. (1998) J. Clin. Invest. 102, 115-123
Hill R. G. Teshima T., Gerbita A., Pan L., Cooke K. R., Brinson Y. S., Crawford J. M., Ferrara J. L. M. (1999) J. Clin. Invest. 104, 459-467
Krijanovsky, O. I., Hill, G. R., Cooke, K. R, Teshima, T., Brinson, Y. S., Ferrara, J. L. M. Blood (1999); 94:825-31
Morse H. C., Shen F. W., Hamerling U. (1987). Immunogenetics. 25, 71
O'Keeffe, M., Hochrein, H., Vremec, D., Pooley, J., Evans, R, Woulfe, S., Shortman, K. Blood (2002); 99(6):2122-30
Pan L., Delmonte J., Jalonen C. K., Ferrara J. L. M. (1995). Blood. 86, 4422-4429
Pan L., Teshima T., Hill G. R., Bungard D., Brinson Y. S., Reddy V. S., Cooke K. R, Ferrara J. L. M. (1999) Blood 93, 4071-4078
Snover, D. C., Weisdorf, S. A., Ramsay, N. K., McGlave, P., Kersey, J. H. Hepatology (1984); 4:123-130
Snover, D. C., Weisdorf, S. A., Vercellotti G. M., Rank, B., Hutton, S., McGlave, P. Human Pathol. (1985); 16:387-392

Vremec D., Pooley J. Hochrein H., Wu L., Shortman K. (2000) J. Immunol. 164, 2978

TABLE I
CD4⁺ T cell responses in primary culture

| | control T | G-CSF T | ProGP-1 T |
|---|---|---|---|
| Cpm | 72.5 ± 4.6 | 55.8 ± 4.2 | 61.6 ± 4.8 |
| IFNγ | 31.1 ± 3.1 | 37.5 ± 3.5 | 35.7 ± 1.1 |
| IL-4 | 523 ± 34 | 2064 ± 81* | 1221 ± 120* |
| IL-10 | 383 ± 191 | 917 ± 72* | 935 ± 145* |

Naïve B6 (H2^{b}) mice received control diluent, G-CSF or ProGP-1 as described in Methods.
Splenic CD4⁺ T cells were purified by magnetic separation or FACS (as described in Methods) and stimulated in primary culture by plate-bound CD3 and CD28 (both at 10 μg/ml).
Results represent mean ± SE of triplicate wells.
*P < 0.05 vs control T cells. Proliferative responses (×10³) were measured by ³H incorporation.
IFNγ (U/ml), IL-4 (pg/ml) and IL-10 (pg/ml) were determined in culture supernatants by ELISA.

TABLE II
Ex vivo donor T cell responses.

| | control T | G-CSF T | ProGP-1 T |
|---|---|---|---|
| CD4+T cells: | | | |
| CD3/CD28 | | | |
| Cpm | 77575 ± 2605 | 45665 ± 1636* | 32640 ± 1366* |
| IFNγ | 591 ± 12 | 285 ± 6* | 299 ± 44* |
| IL-4 | 3016 ± 62 | 1042 ± 44* | 810 ± 40* |
| IL-10 | 1966 ± 87 | 432 ± 32* | 529 ± 18* |
| MLC | | | |
| (anti-H2^{d}) | | | |
| Cpm (S.I) | 3914 ± 649 (6.0) | 1866 ± 177 (2.0)* | 1183 ± |
| | | | 128 (1.0)* |
| IFNγ | 46 ± 2 | 31 ± 1* | 11 ± 1 |
| CD8 + T Cells | | | |
| Cytotoxicity | | | |
| LU₂₀ | 3.7 | 3.7 | 5.4 |
| LU₁₀ | 8.3 | 7.7 | 10.0 |

Mice were transplanted as described in Methods. Seven days later, splenic CD4⁺ T cells were purified by magnetic separation or FACS and stimulated in culture by plate-bound CD3 and CD28 (both at 10 μg/ml) or alloantigen (irradiated B6D2F1 peritoneal macrophages).
Ex vivo responses to alloantigen were determined in MLC.
Results represent mean ± SE of triplicate wells and one of three similar experiments.
*P < 0.05 vs control T cells. Proliferative responses (×10³) were measured by ³H incorporation.
Stimulation index (S.I) is the proliferation to alloantigen/unstimulated cultures.
IFNγ(U/ml), IL-4 (pg/ml) and IL-10 (pg/ml) were determined in culture supernatants by ELISA.
IL-4 (pg/ml) and IL-10 (pg/ml) were at or below the level of detection in MLC cultures and no cytokines were detectable from unstimulated cultures.
Cytotoxicity is presented as lytic units (the effector: target ratio at which 10% and 20% specific lysis was recorded). Lysis to donor type targets was <2%. Data is one of three experiments in which consistent differences in cytotoxicity could not be demonstrated between groups.

1. A method of modulating the immunoactivity of an immunocompetent graft, said method comprising contacting said graft tissue with an effective amount of progenipoietin or functional derivative, homologue, analogue, chemical equivalent or mimetic thereof.

2. The method according to claim 1 wherein said modulation is down-regulation.

3. The method according to claim 2 wherein said progenipoietin is progenipoietin-1.

4. The method according to claim 2 or 3 wherein said graft is an allogeneic graft.

5. The method according to any one of claims 1-4 wherein said graft is a bone marrow graft, spleen cell graft or stem cell graft.

6. A method of generating a population of protective immune cells, said method comprising culturing an immunocompetent population of cells with an effective amount of progenipoietin or functional derivative, homologue, analogue, chemical equivalent or mimetic thereof, wherein said protective immune cells down-regulate the immunoactivity of said immunocompetent cells.

7. The method according to claim 6 wherein said progenipoietin is progenipoietin-1.

8. The method according to claim 6 or 7 wherein said immunoactivity is directed to an allogeneic target cell population.

9. The method according to claim 7 or 8 wherein said immunocompetent population of cells is a bone marrow population, spleen cell population or stem cell population.

10. The method according to any one or claims 69 wherein said protective immune cells are CD4⁺T cells.

11. A method for the prophylactic and/or therapeutic treatment of a condition characterised by the aberrant, unwanted or otherwise inappropriate immunoactivity of an immunocompetent graft, said method comprising contacting said graft tissue with an effective amount of progenipoietin or functional derivative, homologue, analogue, chemical equivalent or mimetic thereof, for a time and under conditions sufficient to down-regulate the immunoactivity of said graft.

12. The method according to claim 11 wherein said progenipoietin is progenipoietin-1.

13. The method according claim 12 wherein said graft is an allogeneic graft.

14. The method according to any one of claims 11-13 wherein said graft is a bone marrow graft, spleen cell graft or stem cell graft.

15. The method according to claims 13 or 14 wherein said condition is graft versus host disease.

16. The method according to claim 15 wherein said graft is pre-treated with progenipoietin.

17. A method for the therapeutic and/or prophylactic treatment of a condition characterised by the aberrant, unwanted or otherwise inappropriate immunoactivity of an allogeneic immunocompetent graft in a subject, said method comprising administering to said mammal an effective number of the protective immune cells generated in accordance with the method of any one or more of claims 6-10 prior to, subsequently to or concomitantly with said graft.

18. The method according to claim 17 wherein said progenipoietin is progenipoietin-1.

19. The method according claim 17 or 18 wherein said graft is an allogeneic graft.

20. The method according to claim 19 wherein said condition is graft versus host disease.

21. The method according to claim 18-20 wherein said graft is a bone marrow graft, spleen cell graft or stem cell graft.

22. The method according to any one of claims 17-21 wherein said graft is pre-treated with progenipoietin.

23. Use of progenipoietin or functional derivative, homologue, analogue, chemical equivalent or mimetic thereof in the manufacture of a composition for the treatment of a graft wherein said treatment down-regulates the immunoactivity of said graft.

24. Use according to claim 23 wherein said progenipoietin is progenipoietin-1.

25. Use according to claim 23 or 24 wherein said graft is an allogeneic graft.

26. Use according to claim 23, 24 or 25 wherein said graft is a bone marrow graft, spleen cell graft or stem cell graft.

27. Use of progenipoietin or functional derivative, homologue, analogue, chemical equivalent or mimetic thereof in the manufacture of a medicament for the treatment of a condition characterised by the aberrant, unwanted or otherwise inappropriate immunoactivity of an immunocompetent graft wherein said treatment down-regulates the immunoactivity of the graft.

28. Use according to claim 27 wherein said progenipoietin is progenipoietin-1.

29. Use according to claim 27 or 28 wherein said graft is an allogeneic graft.

30. Use according to claim 29 wherein said condition is graft versus host disease.

31. Use according to claim 27-30 wherein said graft is a bone marrow graft, spleen cell graft or stem cell graft.

32. Use according to claim 27-31 wherein said graft is pre-treated with progenipoietin.
